(19) United States
(12) Patent Application Publication
(10) Pub. No.: US 2020/0222296 A1
(43) Pub. Date: Jul. 16, 2020

(54) SKIN CELL ENERGY REGENERATION BY FEEDING ADENOSINE TRIPHOSPHATE (ATP) THROUGH TOPICAL DELIVERY ROUTE

(71) Applicants: Steve Sheng Lee, Sandy, UT (US); Francis Wong, Singapore (SG)

(72) Inventors: Steve Sheng Lee, Sandy, UT (US); Francis Wong, Singapore (SG)

(21) Appl. No.: 16/743,944

(22) Filed: Jan. 15, 2020

Related U.S. Application Data
(60) Provisional application No. 62/918,130, filed on Jan. 16, 2019.

(51) Int. Cl. A61K 8/55; A61K 8/49; A61K 8/44; A61K 8/31; A61K 8/65; A61K 8/73; A61K 8/365; A61K 8/9789; A61Q 19/08

(52) U.S. Cl. CPC A61K 8/55; A61K 8/4973; A61K 8/445; A61K 8/31; A61Q 19/08; A61K 8/735; A61K 8/365; A61K 8/9789; A61K 8/65

(57) ABSTRACT

According to one embodiment of the invention, there is a method for the utilization of adenosine 5′-triphosphate (will be called ATP in the context) in the formulation of a topical skin care composition. The method includes the step of topical delivery methods of adenosine triphosphate (ATP) that is used to feed exogenous mitochondria in the skin cell to turn on this molecular battery. Thus the skin cells activated/stimulated to generate even more energy.

### CROSS-REFERENCE TO RELATED APPLICATIONS

This invention claims priority, under 35 U.S.C. § 120, to the U.S. Provisional Patent Application No. 62/918,130 to Lee et al. filed on Jan. 16, 2019, which is incorporated by reference herein.

### BACKGROUND OF THE INVENTION

### Field of the Invention

The present invention relates to skin care compositions, specifically a topical skin care composition including adenosine triphosphate.

### Description of the Related Art

Skin care has been practiced before biblical documentation, dating back to the Ancient Egyptians. Cleopatra, the exotic beauty of an Egyptian ruler was known for her skin care regimen, and is said to have discovered some of the first anti-aging products. Olive oil, milk, egg, etc. were used thousands of years ago in a bath or as a skin mask. Skin care has evolved through the years with every generation being eager to slow, prevent or reverse the aging process.

Countless skin care products are commercially available for beautification of the skin and to fight wrinkle formation. For example, U.S. Pat. No. 4,938,969 discloses a composition for reducing the depth or intensity of fine wrinkles in skin affected by intrinsic or photo-induced aging. The topical formulation described by U.S. Pat. No. 4,938,969 is comprised of ascorbic acid, tyrosine and a non-toxic zinc salt and is preferably formulated in a hydrophilic ointment or cream base. This composition is reported to be effective for the treatment of aging or photo-damaged skin and in reducing wrinkles.

The effectiveness of all skin care products is normally contingent upon delivery of the active ingredients therein through the stratum corneum and viable epidermis into the dermis layer of the skin structure. This is because the active ingredients in the skin care product cannot be effective unless they penetrate through the dead layers of skin tissue and into the dermis layer of living skin cells. This is normally a difficult proposition for water soluble active ingredients, such as ascorbic acid, because the stratum corneum is a good water barrier. The stratum corneum and viable epidermis act to protect the body by holding water therein to prevent dehydration and by keeping external water which is frequently contaminated out of the body.

Some improvements have been made in the field. Examples of references related to the present invention are described below in their own words, and the supporting teachings of each reference are incorporated by reference herein:

U.S. Pat. No. 8,435,547, issued to Blass et al., discloses a skin care composition is useful for augmenting cellular metabolism in skin cells and thereby enhancing the regulation of intracellular signaling. The composition comprises a primer for skin cell mitochondrial function, such as a Krebs cycle intermediate, a precursor of a Krebs cycle intermediate, salts or esters thereof, or combinations thereof. The composition may also include antioxidants for free radical regulation and a pharmaceutically acceptable topical vehicle, such as an emollient base for skin health. A method for stimulating the mitochondrial activity of skin cells comprises administering to the skin of a person in need thereof a composition as described herein.

U.S. Pat. No. 7,629,329, issued to Lee et al., discloses compositions having an effective amount of Adenosine Triphosphate (“ATP”) sufficient to effect intracellular and extracellular concentrations of ATP in a mammal to improve anaerobic exercise capacity by increasing muscle size and/or strength and methods for using the same. Preferably, a gastric acid secretion inhibitory coating is applied to the effective amount of ATP in a manner that protects the ATP from degradation by gastric juices. ATP compositions of the present invention may be administered in nutraceutical or functional food dosage forms, including oral and non-oral delivery forms. In addition, the effective amount of ATP maybe combined with amino acids, botanicals, functional foods, herbals, nucleotides, nutraceuticals, nutrients, pharmaceuticals, proteins, and/or vitamins in an effort to enhance the targeted activity of the composition.

U.S. Pat. No. 9,968,546, issued to Giampapa et al., discloses a topical composition for anti-aging and anti-UV damage treatment of the skin includes water soluble extract of Uncaria species, Arabidopsis thaliana extract, alpha lipoic acid, dimethylethanolamine, tetrahexyldecyl ascorbate, dimethyl sulfoxide, Glycyrrhiza Glabra (licorice) root extract, methylsulfonylmethane, phytosterols, D-ribose, tocotrienol, tocopherol, glucosamine hydrochloride, Pisum sativum extract; silicates of sodium, magnesium and aluminum, and a dermatologically acceptable liposomal delivery medium. The composition is useful in a method using it for anti-aging skin treatment effecting DNA repair in both the nucleus and the mitochondria. The topical composition can be applied on the skin of a person daily, and can also be applied in a dermal infusion treatment, with or without micro-dermal abrasion or skin suction. Topical application of the composition achieves effective reduction of pigmentation spots, degree of winkles, and improvement of skin color, tone and turgidity, particularly of the eyelid and facial areas.

U.S. Patent Application Publication No.: 2006/0216251, by Morariu, discloses a topical composition comprising a lipoic acid, a carnitine, and a carnosine in a suitable vehicle for topical application and a method for treating skin is provided. The present compositions are useful in improving the appearance of aged skin characterized by wrinkles and loss of elasticity. Preferred components include R-lipoic acid or R-dihydrolipoic acid, acetyl-1-carnitine, and 1-carnosine.

U.S. Patent Application Publication No.: 2010/0291190, by Giampapa, discloses a topical composition for anti-aging treatment of the skin includes water soluble extract of Uncaria species, Arabidopsis thaliana extract, lipoic acid, dimethylethanolamine, tetrahexyldecyl ascorbate, dimethyl sulfoxide, Glycyrrhiza Glabra(Licorice) root extract, methyl sulfonylmethane, phytosterols, D-ribose, tocotrienol, tocopherol, glucosamine hydrochloride, Pisum sativum extract; Bambusa vulgaris extract, and a dermatologically acceptable liposomal delivery medium. Further disclosed is a method using the topical composition for anti-aging skin treatment utilizing DNA repair in both the nucleus and the mitochondria. The topical composition can be applied on the skin of a person daily, and can also be applied in a dermal infusion treatment, with or without micro-dermal abrasion or skin suction. Topical application of the composition achieves effective reduction of pigmentation spots and degree of winkles, and improvement of skin color tone.

U.S. Patent Application Publication No.: 2010/0331274, by Gupta et al., discloses This invention relates to certain chirally-correct mitoprotectant phosphorylated heterocyclic agents [formula (I)], which are useful for the treatment of dermatological disorders that include challenged skin from cancer, diabetes, radiation treatments, chemotherapy, and sun-burn; mitochondrial dysfunction; age spots; acne, loss of cellular antioxidants; skin changes associated with aging including collagen loss, loss of skin pliability, loss of skin suppleness, skin wrinkles and fine lines, oxidation, damage from radiation, damage from free radicals, and damage from UV; dry skin; xerosis; ichthyosis; dandruff; brownish spots; keratoses; melasma; lentigines; liver spots; skin pigmentation including pigmented spots, dark circles under the eyes, darkened skin, and blemishes; oily skin; warts; eczema; pruritic skin; psoriasis; inflammatory dermatoses; topical inflammation; disturbed keratinization; scalp dryness; skin depigmentation, and combinations thereof;

The inventions heretofore known suffer from a number of disadvantages which include being limited in use, being too expensive, being difficult to use, having to be ingested, having to be injected, being ineffective, being inefficient, being costly, having to be reapplied multiple times daily, not being able to activate the skin cell via turning on the molecular battery of skin cell, mitochondria, not being able to absorb all the nutrients, being limited in use, being limited in benefits. Without turning on the skin energy furnace the nutrients in the products will not or very poorly absorb utilized by our skin.

What is needed is a topical skin care composition that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available skin care compositions. Accordingly, the present invention has been developed to provide a topical skin care composition including adenosine triphosphate to increase skin cell energy regeneration.

According to one embodiment of the invention, there is a topical skin care composition that may include about 0.1 weight percent to about 10 weight percent of adenosine triphosphate. The composition nay include about 1 weight percent to about 20 weight percent ascorbic acid. The composition may also include about 1 weight percent to about 10 weight percent phenylalanine. The skin care composition may include about 1 weight percent to about 10 weight percent tyrosine. The composition may include about 0.5 weight percent to about 5 weight percent of a retinol.

The topical skin care composition may include a retinol; wherein the retinol may be Retinyl Palmitate or Retin-A. The composition may also include about 0.1 weight percent to about 10 weight percent of hydrolyzed gamat collagen. In addition, the skin care composition may include about 0.1 weight percent to about 10 weight percent of hyaluronate. The composition may also include about 0.1 weight percent to about 10 weight percent of alpha hydroxy acid. The topical skin care composition may include about 0.1 weight percent to about 10 weight percent of shea.

According to one embodiment of the invention, there is a method of reenergizing human skin affected by intrinsic aging, environment pollutants and/or UV light-induced aging, that may include the step of topically applying a topical formulation to the skin. The topical formulation may include a topical skin care composition that may include adenosine triphosphate. The composition may include: ascorbic acid, phenylalanine, tyrosine, retinol (along with Retinyl Palmitate and Retin-A) is one form of a retinoid, hydrolyzed gamat collagen, hyaluronate, alpha hydroxy acid, and/or shea.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a top plan cross-sectional view of a skin cell, according to one embodiment of the invention;

FIG. 2 is a top plan cross-sectional view of a mitochondria, according to one embodiment of the invention; and

FIG. 3 is a method diagram of skin cell energy regeneration by feeding adenosine triphosphate through topical delivery route, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

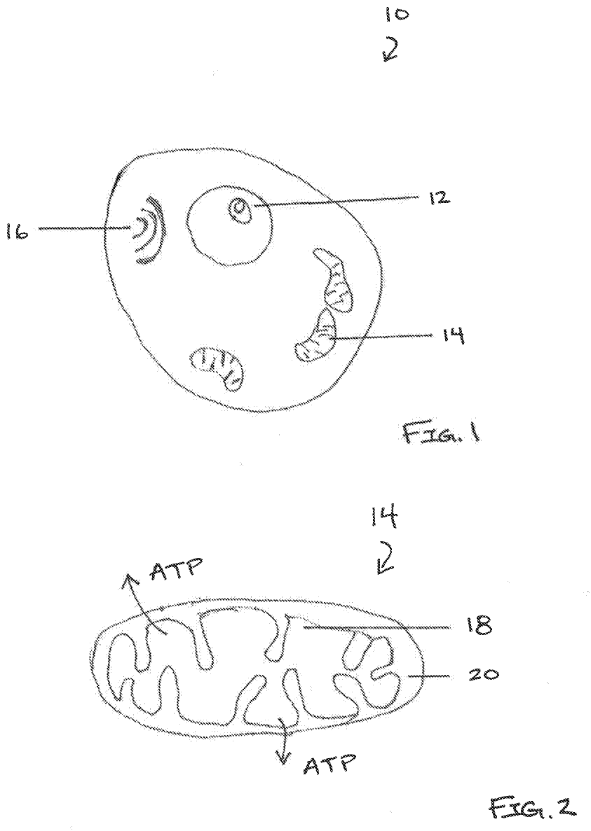

FIG. 1 is a top plan cross-sectional view of a skin cell, according to one embodiment of the invention. The illustrated skin cell 10 includes mitochondria 14, nucleus 12, and golgi 16.

The illustrated skin cell 10 is activated by adenosine triphosphate (ATP) through topical delivery; wherein the adenosine triphosphate interacts with the mitochondria 14 of the skin cell 10 to provide energy regeneration in the skin cell 10 to soothe the pain of sunburns and in the treatment of red, irritated, dry, cracked or itchy skin. An increase of ATP to the skin cell will increase the production of ATP from the mitochondria 14 within each skin cell 10

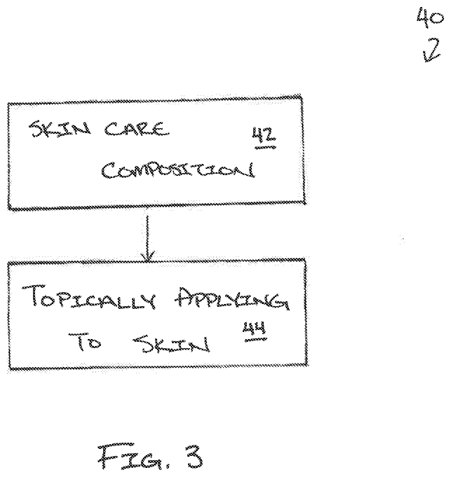

FIG. 2 is a top plan cross-sectional view of a mitochondria, according to one embodiment of the invention. The illustrated mitochondria 14 includes a matrix 18 and a membrane 20.

The illustrated mitochondria 14 generates adenosine triphosphate (ATP) from the matrix 18; wherein the ATP travels through the membrane 20 of the mitochondria 14 to the skin cell 10. Providing ATP to the skin cell, topically, gives the mitochondria 14 a boost in generating even more ATP to increase skin cell energy, characteristics, touch, tone, feel, and other qualities.

FIG. 3 is a method diagram of skin cell energy regeneration by feeding adenosine triphosphate through topical delivery route, according to one embodiment of the invention. The illustrated method of skin cell energy regeneration by feeding adenosine triphosphate through topical delivery route 40 includes the step of providing a skin care composition 42 and a step of topically applying the skin care composition 44.

According to one embodiment of the invention, there is a skin delivery system and dosage form, that includes a topical skin care composition that is used to rejuvenate skin that has been damaged by exposure to environmental pollutants, intrinsic factors, ultraviolet rays, or which has simply been affected over the years by physical and mental aging. The skin care composition is used to slow the rate of photo-induced aging to maintain beautiful skin tone and texture over the years. The composition inhibits the formation of wrinkles and in some cases reduces the depth of existing wrinkles or eliminates them entirely. In some cases, the skin care composition of this invention also lightens age spots and other types of blemishes associated with aging.

According to one embodiment of the invention, there is a topical skin care composition that is non-irritating and is used to soothe the pain of sunburns and in the treatment of red, irritated, dry, cracked or itchy skin. The skin care composition is used in treating atopic dermatitis, psoriasis, and ichthyosis by moisturizing the skin. The skin care composition of this invention is applied to the face, décollete areas, and/or hands of a consumer. However, the composition may be generally used anywhere on the skin. For instance, the topical skin care composition may be applied to feet, chest, back, legs, ankles, arms, and/or wrists as desired.

According to one embodiment of the invention, there is a skin care composition that is based upon the discovery that an energy unit of adenosine triphosphate (ATP) generated in mitochondria of a skin cell, Retinol (along with Retinyl Palmitate and Retin-A) is one form of a retinoid, Shea for dull skin, natural ascorbic acid as a skin antioxidant base including hydrolyzed gamat collagen, hayaluronic sodium as a skin hydrating agent, and lactic acid in milk used to improve the penetration of the above ingredients.

According to one embodiment of the invention, there is a method of reenergize human skin affected by aging, environment pollutants and/or UV light-induced aging. The method includes the step of topically applying a topical skin care composition or formulation to the skin, wherein the topical skin care composition is comprised of about 0.1 weight percent to about 10 weight percent adenosine triphosphate (ATP), about 1 weight percent to about 20 weight percent ascorbic acid, about 1 weight percent to about 10 weight percent selected from the group consisting of phenylalanine and tyrosine, about 0.5 weight percent to about 5 weight percent of a Retinol (along with Retinyl Palmitate and Retin-A) is one form of a retinoid, about 0.1 weight percent to about 10 weight percent of Hydrolyzed Gamat collagen, about 0.1 weight percent to about 10 weight percent of Hyaluronate, about 0.1 weight percent to about 10 weight percent of Alpha Hydroxy Acid, and about 0.1 weight percent to about 10 weight percent of Shea.

Only in the past one to two decades have the roles of adenosine triphosphate (ATP) and its major metabolic product, adenosine, began to emerge as a powerful physiological, extracellular regulator of intravascular, extravascular and central nervous system active agent. These roles are attracting significant attention within the field of pharmaceutical development. Extracellular ATP has been shown to regulate cardiac function, vascular tone, skin/muscle cellular activities, neurotransmission and liver metabolism. Extracellular ATP is produced by specific release of ATP from a mitochodria and the ATP interacts with families of purinergic receptors present on the plasma membrane of virtually every skin cell.

Extracellular ATP is the source of extracellular adenosine, which interacts with its own specific receptors around skin cells. In the human body adenosine and ATP receptors are depicted by two major classification; they are P1 and P2 receptors. The adenosine receptors (P1) is further subdivided into A1, A2alpha, A2beta and A3, which in turn are capable of activating phospholipase C (A1, A2beta, A3). That again, regulates adenylate cyclase function (A2alpha, A2beta) and modulates ion channels activities (A1, A2beta, A3). The ATP receptors (P2) belong to a subgroup that acts via G-protein coupled receptors (P2Y) and a subgroup of intrinsic ion channels (P2X). All these mechanisms and receptors work to maintain skin cell energy at their perfect and optimal coordination. This is the ideal situation for healthy and brilliant skin; however most of the time, skin cells suffer damage or dysfunction from environment pollutants, UV sun light, intrinsic coordination shortfalls, aging, and some other environmental or hereditary factors. So, therefore supplementation of ATP through topical delivery via a formulation that is amicable to skin cells is preferred.

According to one embodiment of the invention, there is a topical skin care composition composed of purified water, white petrolatum, cetearyl alcohol and ceteareth-20, sorbitol solution, propylene glycol, dimethicone, glyceryl monostearate. Dimethicine is also known as also known as dimethylpolysiloxane.

According to one embodiment of the invention, there is a method of reenergize human skin affected by intrinsic aging, environment pollutants and/or UV light-induced damage/aging. The method includes the step of topically applying a topical formulation to the skin, wherein the topical formulation is comprised of about 0.1 weight percent to about 10 weight percent ATP; about 1 weight percent to about 20 weight percent ascorbic acid; about 1 weight percent to about 10 weight percent selected from the group consisting of phenylalanine and tyrosine; about 0.5 weight percent to about 5 weight percent of a Retinol (along with Retinyl Palmitate and Retin-A) is one form of a retinoid; about 0.1 weight percent to about 10 weight percent of Hydrolyzed Gamat collagen; about 0.1 weight percent to about 10 weight percent of Hyaluronate; about 0.1 weight percent to about 10 weight percent of Alpha Hydroxy Acid; about 0.1 weight percent to about 10 weight percent of Shea; and about 0.1 weight percent to about 10 weight percent of Alpha-tocopherol.

According to one embodiment of the invention, there is a topical skin care composition that achieves the following skin improvement: acting on skin peripheral circulation, acting on skin cell energy generation, stimulating the production of dermal ATP, scavenging free-radical, de-pigmenting, and moisturizing bioactivities.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

### Example 1

In this example a topical skin care composition was made utilizing 85% of the base (oil-in-water emulsion that consists of purified water, white petrolatum, cetearyl alcohol and ceteareth-20, sorbitol solution, propylene glycol, dimethicone, also known as dimethylpolysiloxane, glyceryl monostearate,) ATP, 5 weight percent, Ascorbic acid 5 weight percent, Retinol (along with Retinyl Palmitate and Retin-A, 5 weigh percent.

The topical skin care composition is soothing when applied to dry skin and has good moisturizing characteristics. The composition also provides a brilliant glow to skin due to enhanced skin cell energy due to the activity of ATP. This skin care composition has very little odor without adding fragrance.

### Example 2

In this example formulation of a topical skin care composition is made using the procedure described in Example 1 except Gamat collagen was added to the formulation replacing the 5 weight percent of Ascorbic Acid. The topical skin care composition formulation made is soothing when applied to dry skin and has good moisturizing characteristics. The composition also provides a brilliant glow to skin due to enhanced skin cell energy due to the activity of ATP. The skin care composition has healing properties and unique glycoprotein content on damaged skin surface caused by intrinsic aging, environment pollutants and/or UV light-induced damage/aging,

### Example 3

A healing and moisturizing skin care composition for damaged and dry skin is demonstrated by this experimental formula. In this example a topical skin care composition is made utilizing 75% of the base (oil-in-water emulsion that consists of purified water, white petrolatum, cetearyl alcohol and ceteareth-20, sorbitol solution, propylene glycol, dimethicone, also known as dimethylpolysiloxane, glyceryl monostearate,) Gamat Glycoprotein 10 weight percent, ATP, 5 weight percent, Ascorbic acid 5 weight percent, Retinol (along with Retinyl Palmitate and Retin-A, 4 weigh percent and 1% Sodium Hyaluronate. The composition is mixed with a mixer running at about 60° C. for about 20 minutes to attain a uniform creamy state. No Fragrance is added.

Female subjects applied this skin composition to their faces without any other skin care products. The composition is reported to have good moisturizing characteristics and is further reported to provide the facial skin to which it was applied with a brilliant glow. Some blemish areas are rejuvenated after a few applications.

### Example 4

In this example a topical skin care composition is made by utilizing 85% of the base (oil-in-water emulsion that consists of purified water, white petrolatum, cetearyl alcohol and ceteareth-20, sorbitol solution, propylene glycol, dimethicone, also known as dimethylpolysiloxane, glyceryl monostearate,) ATP, 5 weight percent, Ascorbic acid 5 weight percent, Retinol (along with Retinyl Palmitate and Retin-A, 2 weigh percent, Alpha Hydroxy Acid, 2 weight percent and Shea, 1 weight percent. No Fragrance is added. Subjects experienced brilliant skin glow, and lighter shade after 3 weeks use.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A topical skin care composition to increase adenosine triphosphate generation, comprising about 0.1 weight percent to about 10 weight percent of adenosine triphosphate.

2. The composition of claim 1, wherein the composition further comprises about 1 weight percent to about 20 weight percent ascorbic acid.

3. The composition of claim 1, wherein the composition further comprises about 1 weight percent to about 10 weight percent phenylalanine.

4. The composition of claim 1, wherein the composition further comprises about 1 weight percent to about 10 weight percent tyrosine.

5. The composition of claim 1, wherein the composition further comprises about 0.5 weight percent to about 5 weight percent of a retinol.

6. The composition of claim 5, wherein the retina is Retinyl Palmitate.

7. The composition of claim 5, wherein the retinol is Retin-A.

8. The composition of claim 1, wherein the composition further comprises about 0.1 weight percent to about 10 weight percent of hydrolyzed gamat collagen.

9. The composition of claim 1, wherein the composition further comprises about 0.1 weight percent to about 10 weight percent of hyaluronate.

10. The composition of claim 1, wherein the composition further comprises about 0.1 weight percent to about 10 weight percent of alpha hydroxy acid.

11. The composition of claim 1, wherein the composition further comprises about 0.1 weight percent to about 10 weight percent of shea.

12. A topical skin care composition to increase the generation of adenosine triphosphate within a skin cell, comprising:
a) ATP;
b) ascorbic acid;
c) phenylalanine;
d) tyrosine;
e) a Retinol;
f) hydrolyzed gamat collagen;
g) hyaluronate;
h) alpha hydroxy acid; and
i) shea.

13. A method of reenergizing human skin affected by intrinsic aging, environment pollutants and/or UV light-induced aging, including the step of topically applying a topical formulation to the skin, wherein the topical formulation comprises a topical skin care composition including adenosine triphosphate.

14. The method of claim 13, wherein the skin care composition further comprises:
a) ascorbic acid;
b) phenylalanine;
c) tyrosine;
d) retinol (along with Retinyl Palmitate and Retin-A) is one form of a retinoid;
e) hydrolyzed gamat collagen;
f) hyaluronate,
g) alpha hydroxy acid; and
h) shea.
